(19) United States
(12) Patent Application Publication
(10) Pub. No.: US 2002/0119437 A1
(43) Pub. Date: Aug. 29, 2002

(54) Method of preparing and processing transplant tissue

(76) Inventors: Jamie M. Grooms, Alachua, FL (US); C. Randal Mills, Alachua, FL (US)

Correspondence Address: VAN DYKE & ASSOCIATES, P.A., 1630 HILLCREST STREET, ORLANDO, FL, 32803, US

(21) Appl. No.: 09/960,256

(22) Filed: Sep. 20, 2001

Related U.S. Application Data
(60) Provisional application No. 60/234,013, filed on Sep. 20, 2000.

(51) Int. Cl. A01N 1/02; C07K 14/78

(52) U.S. Cl. 435/2; 530/356

(57) ABSTRACT

Disclosed is a method for processing collagen-based tissues or organs to substantially decellularize said collagen-based tissues or organs prior to implantation into a recipient in need thereof. Preferably, the method significantly reduces or eliminates immunogenicity of the tissue or organ such that upon transplantation, the tissue or organ is not rejected by the recipient's immune system. The method includes removing the tissue from a donor, processing the tissue to remove substantially all of the cells of the tissue or organ, and processing of the collagen scaffold for storage. The method further includes repopulating the collagen scaffold through seeding with stem cells for implantation into recipient.

### FIELD OF THE INVENTION

This invention is directed to a method for processing an organ, tissue, joint, and the like for use in transplantation, and to the tissue thereby produced.

### BACKGROUND OF THE INVENTION

Known methods and procedures for tissue and organ transplantation have many drawbacks, such as inflammation, rejection by host, scarring and calcification of the transplant tissue. Typically, immunosuppressive compounds must be administered to recipients of known transplant tissues. However, the essential daily doses of immunosuppressant drugs eventually become inactive, and concomitant susceptibility to bacterial, viral and other infections are significant additional drawbacks to immunosuppressive treatment. Frequently, new transplants are required after a few months or years, since initial transplants are often rejected by the recipient's body (graft versus host disease).

In the rapidly growing field of tissue and organ transplantation, efforts have been made to reduce immunogenicity of transplantable tissue and increase acceptance rates in recipients. The following patent publications disclose various methods for decellurizing, storing and repopulating collagen-based tissues: U.S. Pat. Nos. 5,613,982; 5,336,616; 5,595,571; 5,632,778; 5,192,312; 5,893,888; 5,855,617 and WO99/41981. The disclosure of each of these patent publications is hereby incorporated by reference. In U.S. Pat. No. 6,027,743, hereby incorporated herein by reference, methods and implants were described wherein total joint replacement was disclosed upon treatment of a harvested cadaveric joint with ethanol for many hours, followed by freeze-drying. The problem with that technology is that the tissue, while presumably largely “devitalized”, is not free from significant quantities of immunogenic cellular materials, both on the implant surfaces, and within the interstices of the bone. Accordingly, while that patent purports to provide an implant that is “sufficiently non-antigenic to prevent graft rejection in vivo”, the patent does not address the concern, particularly when use of xenograft materials is contemplated. It is recognized that implanted xenograft material normally induces a stronger non-self immune response as compared to allograft material. Thus, for xenograft tissues undergoing the procedure of U.S. Pat. No. 6,027,743, it is contemplated that immunogenic cellular components would remain with the “devitalized” freeze-dried harvested material, and these would induce adverse immune responses in the recipient.

Accordingly, despite some advancements in the field of tissue and organ transplantation, convenience and availability of both processing and receiving transplantable tissue remains a problem. There remains a need for a method of treating an entire organ, such that the organ can be made to order for implantation. Traditional allograft or xenograft tissues, including soft and hard organs may require immediate use after tissue recovery. Therefore, a method allowing organs to be made available to order on a convenient schedule would be superior to methods currently known in the field. The present invention meets this need.

### SUMMARY OF THE INVENTION

Disclosed is a method for processing an organ or other collagen-based tissue to reduce immunogenicity for use in transplantation. The method includes removing tissue from a donor, processing the tissue to remove all the cells, and processing of the collagen scaffold for storage. The method further includes repopulating the collagen scaffold through seeding with stem cells or other cells for implantation into a recipient in need thereof.

Accordingly, it is one object of this invention to provide a method for processing a collagen-based tissue or an organ to provide transplantable material with reduced immunogenicity.

Another object of this invention is to provide a method of decellurizing a collagen-based tissue or organ which renders the tissue essentially non-immunogenic.

Another object of this invention is to provide a method of repopulating a collagen-based tissue or organ with non-immunogenic cells.

Additional objects and advantages of the method and implants according to this invention will become apparent from a review of the complete disclosure.

### DESCRIPTION OF THE DRAWINGS

FIG. 1 depicts a schematic first step of the improved process disclosed herein.

FIG. 2 depicts a schematic of a second step of the improved process disclosed herein

FIG. 3 depicts a schematic of a third step of the improved process disclosed herein.

### DETAILED DISCLOSURE OF THE PREFERRED EMBODIMENTS

The method of this invention results in an organ or tissue that can be made to order for use in a transplantation procedure. Tissues to be processed by the present invention include, but are not limited to, tracheal tissue, heart valves, total joints, entire heart, vasculature, soft organs, and any other tissue required for implantation. In one preferred embodiment, the tissue processed by the method of the present invention is a heart. In another preferred embodiment, the tissue processed by the method of the present invention is a knee, shoulder, wrist, ankle, elbow or other joint. The recipient's joint is removed, due to illness or trauma, and the joint prepared according to this invention is implanted according to methods known in the art or which become known hereafter.

According to the method of this invention, the tissue to be treated is removed from a donor and processed to remove all the cells. The remaining collagen scaffold is then “seeded” with non-immunogenic cells including but not limited to stem cells, fetal cells and the like to repopulate the tissue before transplantation into a recipient. Depending on the type of tissue being treated and to be replaced, different stem cells known in the art or which become known hereafter are selected such that appropriate tissues are formed upon implantation into a recipient of the seeded implant.

According to the present invention, a collagen structure is left completely (or nearly so) intact while all cells and cellular debris, lipids and non-collagenous proteins are thoroughly eliminated. A preferred process for use according to this invention is known as the BIOCLEANSE PROCESS™, publication no. WO 00/29037. Applicants provide herein below (part II) further modifications and improvements to this novel process. The process can be used to treat autograft material ex vivo for reimplantation, so that tissues from different donors, whether animal or human, (allogenic or xenogenic), may be cleaned, sterilized and/or decellularized. The process utilizes rapid pressure cycling to achieve penetrating cleaning of tissues. Deep tissue interpenetration by the cleansing solutions is achieved by oscillating the pressure in a chamber while adding and removing various cleansing solvents. The above summarized BIOCLEANSE PROCESS™ for producing an acellular collagen scaffold is more fully disclosed in publication no. WO 00/29037, the entire contents of which is hereby incorporated herein by reference. Alternate methods for decellurizing tissue which can be used in conjunction with the present invention include those disclosed in the following patents; U.S. Pat. Nos. 5,336,616; 5,595,571; and 5,993,844, the disclosures of which are hereby incorporated by reference. Furthermore, U.S. provisional Application No. 60/296,530, directed to a novel method of decellularizing and viral inactivating soft and hard tissues, to which priority is claimed under 35 USC § 119, whose teachings are incorporated herein by this reference.

If the tissue or organ is not to be immediately implanted, the tissue may be processed for storage. Storage may come in the form of a bioreactor, cryopreservation, freezing, chilling, drying, room temperature packaging, or freeze-drying. U.S. Pat. No. 5,336,616 and WO 99/41981 are incorporated herein by reference for disclosure on methods for cryopreservation, freezing and drying of collagen-based tissues.

The remaining collagen scaffold is optionally seeded with non-immunogenic cells before implantation into a recipient. Preferably, the “seeded” collagen scaffold is grown according to known organ perfusion technology. Methods of repopulating collagen-based tissue are disclosed in U.S. Pat. Nos. 5,192,312; 5,863,296 and WO99/60951, the disclosure of each of which is hereby incorporated herein by reference.

Due to the stability and sterility of acellular tissues produced according to this invention, convenient schedules can be established to provide appropriate timing for processing an organ, from the point of receiving an organ from a cadaver to the time when the collagen scaffold is shipped to a hospital for use in transplantation. The establishment of time and availability schedules increases convenience and reliability for both patients and hospitals.

One aspect of the present invention relates to a method of making a substantially intact collagen heart scaffold, which is made to order. According to this aspect of the invention, a patient in need of a heart transplant, upon receiving diagnosis, commissions production of a heart. Upon commission, a heart is removed from a cadaver. The harvested heart is then treated according to the method of this invention, wherein all cells, cellular debris, lipids and non-collagenous proteins are removed. The remaining collagen heart scaffold is seeded with stem cells, myocardial cells, growth factors and the like, and grown in an organ perfusion system. The result is a replacement heart ready for use in heart replacement surgery.

Another aspect of the present invention relates to a method of making a substantially intact knee or other joint. The knee joint is a substantially cleaned portion of a femur, patella and tibia. According to the methodology of this invention, a patient in need of knee replacement surgery commission production of a knee. Upon commission, portions of the femur, patella and tibia are removed from a cadaver. The harvested tissue is then processed according to the method of the present invention, wherein all the cells, cellular debris, lipids and non-collagenous proteins are removed. The remaining collagen scaffold of the knee joint is seeded with cells and repopulated in an organ perfusion system. The result of the present method is a made-to-order replacement knee implant ready for implantation into a patient. Alternatively, upon treatment with appropriate growth factors, the replacement joint may be directly implanted into a patient for in situ revitalization and remodeling.

Another aspect of the present invention relates to a method of making a substantially intact transplantable trachea. A patient in need of tracheal replacement, upon receiving diagnosis, commissions production of a new trachea according to the method of this invention. The necessary portion of the trachea is then removed from a cadaver and treated according to the method of the present invention. The trachea is cleaned to remove all cells, cellular debris, lipids and non-collagenous cells. The remaining collagen scaffold of the trachea is seeded with stem cells. After seeding, the collagen scaffold is processed in an organ perfusion system, wherein new cells are grown to repopulate the trachea. After processing according to the methods of this invention, the harvested trachea is ready for transplantation into a patient.

II. Improvement to Process as Described in WO00/29037

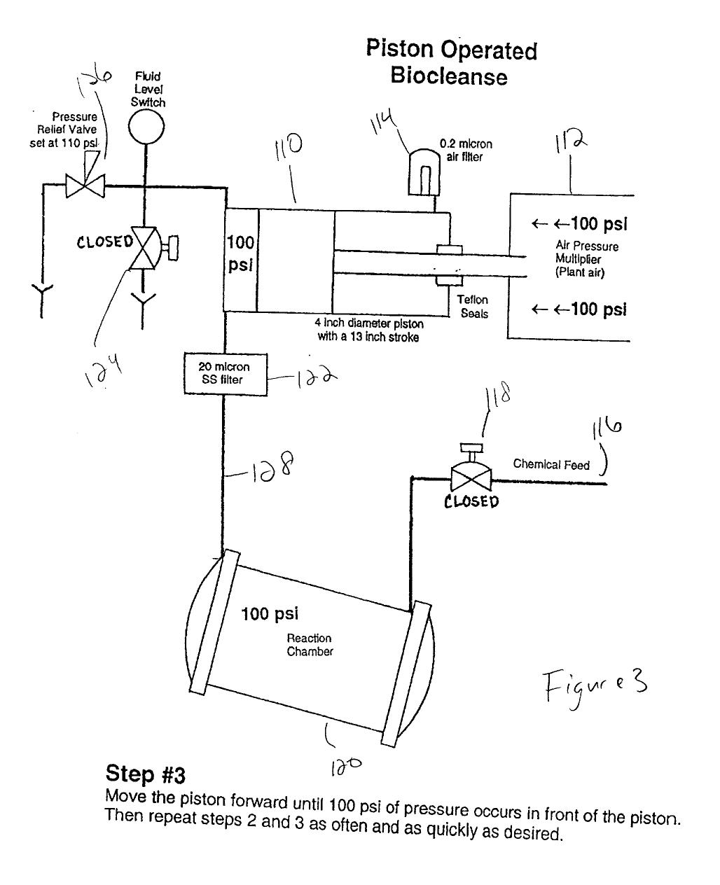

The basic process described in WO 00/29037 is improved by incorporating and implementing the use of a simple single air piston device to provide both the air pressure and the vacuum necessary for the process. The process as described in WO 00/29037 preferably uses a sanitary air filtration system (which can be expensive due to the complex steam in place and integrity testing system required for clean air) and a valving system to provide clean air for preferably 100 psi pressure and another sanitary valving receiver and pump system to provide the vacuum. The valving receiver preferably comprises a large stainless steel tank with anti-foam and cleaning provisions. It is desirable to provide small inexpensive reaction chamber units that can be utilized for “no fluid mixing” single donor processing. Before the teachings of the present invention, configuring small ingle donor reaction chambers would likely require a separate vacuum reservoir, a separate vacuum pump and the associated valving and equipment for each reaction chamber in order to maintain true “no fluid mixing” (i.e. no mixing of fluid among individual reaction chambers) single donor processing. The use of an air piston as conceived of by the inventors provides a novel, inexpensive solution. Air pistons for incorporation into the current process of WO 00/29037 preferably would be commercially available, sanitary, pharmaceutical precision fitted pistons (e.g. Bosch pistons). With the use of an air piston, no large vacuum reservoir, no vacuum pump, and none of the associated sanitary valving would be necessary as disclosed in WO 00/29037 (specifically shown in FIG. 3). Furthermore, the air pistons can also be set to process bone at a much faster rate. An air piston can go through a pressure/vacuum cycle five times a second, while older processes may require up to fifteen seconds to complete one vacuum/vacuum cycle (due to vacuum pump recovery time). Use of the subject air piston configuration would reduce the cycle time from 3 to 4 hours to half an hour per batch. The subject invention can achieve these reductions in cycle times while meeting and even exceeding sterile isolation guidelines followed in the industry.

The previous process, with its foaming reagents (e.g. hydrogen peroxide and the tissue lipids in combination), produces a thick viscous foam (e.g., up to 6.7 gallons of foam at 100 PSI). Since this foam is under pressure initially, it is then transferred to an 80% vacuum. When it accumulates in the vacuum reservoir it becomes very voluminous (6.7 gallons of foam at 100 PSI equates to about 227 gallons of foam at 80% vacuum). This voluminous foam is the primary reason that a series of small single donor reaction chambers would be difficult to connect to a common vacuum reservoir without cross contamination of fluids. If the voluminous foam from one donor chamber comes in contact with the port evacuating another donor chamber, it might be possible that cross contamination could occur. The subject invention, use of an air piston, does not create the semi-permanent voluminous foam sitting in a vacuum receiver; the foam that is created simply is compressed and uncompressed by the air piston. Furthermore, if one of the reagents of the process (e.g. hydrogen peroxide) creates too much foam, it is just vented through a pressure relief valve.

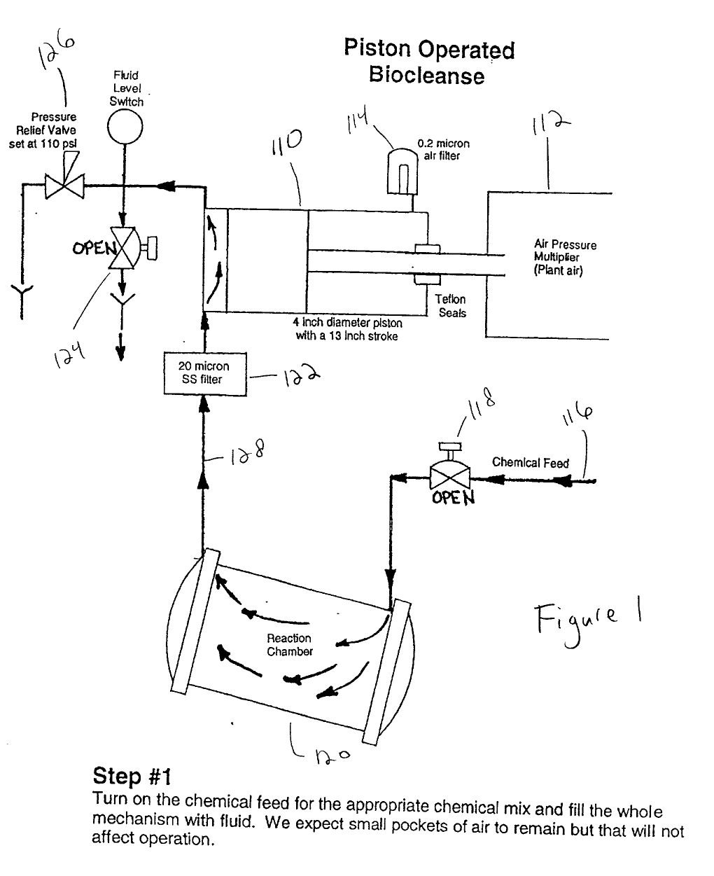

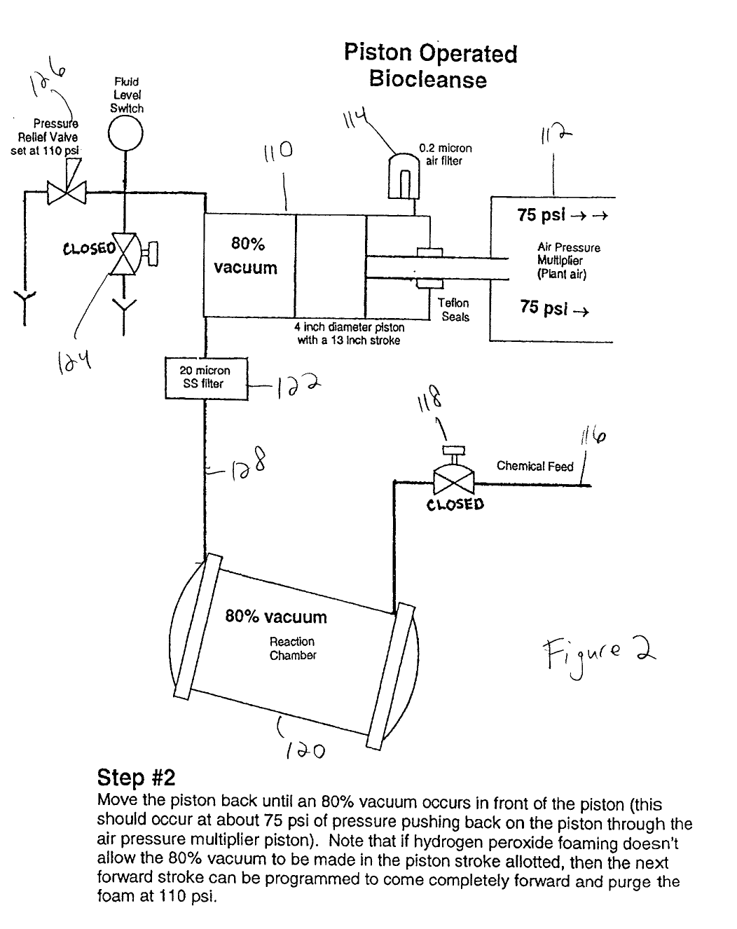

Turning to FIGS. 1-3, the improvement to the BIOCLEANSE described in WO 00/29037 process is shown implementing the air piston 110. The reaction chamber 120 and system is filled with chemical/reagent via the chemical feed 116 until fluid flows through valve 124. Valves 118 and 124 are open during the filling of the system. Upon filling, valves 124 and 118 are closed and the piston 110 is drawn back by the air pressure multiplier 112. Reaction chamber 120 and Air piston 110 are connected via a conduit/line 128. As shown in FIG. 2, preferably a vacuum of 80% is created by movement of the piston 110, which may preferably occur upon application of 75 PSI pushing back on the piston 110. After the vacuum has been applied for a preselected time, the piston 110 is pushed forward via the Air pressure multiplier 112 to produce pressure in the reaction chamber (preferably 100 PSI) as shown in FIG. 3. Steps 2 and 3 are repeated as often and as quickly as desired. As shown, a pressure relief valve 126 is provided in the system to relieve pressure and to purge foam if necessary. Filter 122 is provided along the fluid line of the system to filter debris and other particulate matter. Filter 114 is provided to filter air in or out, as desired, of the piston 110.

Having generally described this invention, including the best mode thereof, the present invention is to be interpreted in light of the appended claims and their equivalents.

III. Preparation of Dermis and Other Tissues

The term “tissue” as used herein includes, but is not limited to, bone, neural tissue, fibrous connective tissue including tendons and ligaments, cartilage, dura, pericardia, muscle, heart valves, veins and arteries and other vasculature, dermis, adipose tissue, or glandular tissue.

“Antimicrobial agents” and/or “viral inactivating agents” as used herein, include, but are not limit cetylpyridinium chloride, hydrogen peroxide, calcium hydroxide, quaternary ammonium compounds, and other such similar compounds as disclosed, e.g., in U.S. Pat. Nos. 6,224,579; 6,175,053; and 5,994,383. Benzalkonium chloride, hydrogen peroxide and calcium hydroxide are preferred agents.

Those skilled in the art will recognize that in view of the teachings herein, the specific examples of treating dermis tissue can be easily adapted and modified for other tissue types. Also, the specific teachings as to particular materials, equipment and steps should not be construed to be limiting, but only refers to one embodiment involving a complex number of components and procedures. Other materials, equipment, and series of steps should be understood to fall within the scope of this invention as described in the specification and as defined by the claims provided below.

MATERIALS AND EQUIPMENT

| | |
|---|---|
| 1.01 MATERIALS | (All materials/equipment shall be autoclaved, irradi- |
| | ated, or sterile filtered, using approved procedures.) |
| A. | Sodium Chloride, aqueous 1M. |
| B. | Benzalkonium Chloride, 1% aqueous. |
| C. | Tween-20 1% +0.5% H₂O_{2.} |
| D. | Saturated Calcium Hydroxide aqueous. |
| E. | EDTA 0.1%, aqueous, pH 8.0. |
| F. | Sodium Monophosphate buffer, pH 7.0. |
| G. | 70% Isopropyl alcohol. |
| H. | Purified Water per USP XXIV. |
| I. | Polypropylene scour wipe. |
| J. | Surgical scalpel blades #10, #20. |
| K. | Large Poly bags. |
| L. | 15cc Centrifuge Tube. |
| M. | Lyophilizer pouches. |
| N. | Tex-wipes. |
| O. | Skin Packaging Kit: Tyvek/Mylar pouch, clear |
| | foil/foil pouch, bar code. |
| P. | D-Test Thio. |
| Q. | D-Test TSB. |
| R. | Tubes-Thio. |
| S. | Tubes-TSB. |
| 1.02 EQUIPMENT | |
| A. | Timer. |
| B. | Thermometer. |
| C. | Graduated cylinder. |
| D. | Sonic. |
| E. | Sonic container (2, 4 liter or larger). |
| F. | Long Forceps. |
| G. | Scoopula. |
| H. | Screen Press. |
| I. | #3 or #4 Scalpel handle. |
| J. | Metzenbaum scissors. |
| K. | Ruler. |
| L. | Graft Templates. |
| M. | Thickness Gauge. |
| N. | Roller System. |
| O. | 2 and 4 liter carboys with lids. |

2.0 Definitions

Dermis—A collagenous tissue supporting the epidermis.

Blunt Dissection—Separating tissues by means not including cutting or tearing.

Pass In—Scanning the bar code when donor material enters a process.

Pass Out—Scanning the bar code when donor material completes a process.

Free Of—An absence of.

Substantially Free Of—A few or if there is a normal pattern, per Tappi Estimation Chart.

3.0 Removal of Epidermis With Sodium Chloride:

3.1 In a class 1000 or better environment, use a thickness starrett snap gauge to measure the thickness of the dermis tissue provided. When determining the thickness of each dermis section, a minimum of three measurements must be made along the length of the tissue. The average of the three measurements is the measurement recorded.

3.2 All donor material measurements are to be recorded on Attachment B.

3.3 Perform a visual inspection of the dermis. Dermis must be:

Free of epidermis, muscle, fat and hair.

Free of, scars, moles, debris, tattoos, and blood. Substantially free of freckles.

Personnel conducting inspection shall record information appropriately on attachment B.

3.4 The average of the pieces used for graft production must be ≧0.7 mm in thickness.

3.4.1 Sections of dermis tissue <0.7 mm in thickness will be placed back into the procurement containers. Place the containers into two poly bags; tie a knot in each bag individually to seal close the open end. Return the containers to freezer. Label appropriately.

3.4.2 Upon completion of the measurements, the dermis tissue ≧0.7-mm in thickness will be placed into a graduated cylinder. Ensure that the tissue is lightly tamped. This is to ensure that the tissue has settled. The tissue once settled has to be below the highest graduation. If it is not then, procure a larger graduated cylinder, transfer tissue to new graduated cylinder and the measure the volume of tissue.

3.4.3 Record the tissue volume in ml on Attachment A.

3.4.4 Multiply the volume of tissues recorded by 10. This calculated value will be the minimum fluid volume used during each step of dermis allograft production.

### Table 1

| | If the recorded tissue | |
|---|---|---|
| | volume is | Processing shall be performed using |
| 1 | Equal to or less than 180 | A 2-liter carboy/sonic beaker |
| | ml | |
| 2 | Greater than 180ml but | A 4-liter carboy/sonic beaker |
| | less than or equal to 360 | |
| | ml | |
| 3 | Greater than 360 ml | Multiple carboys (split the tissue into |
| | | equal parts and designate each carboy/ |
| | | sonic beaker size per 1, 2 and etc) |

3.4.5 Transfer the dermis from the graduated cylinder into the appropriate wide-mouth carboy.

3.4.6 Fill the carboy with the measured amount of fluid, which is at least 10 times the volume of the tissue of filtered 1 M Sodium Chloride and tightly close the cap.

3.4.7 Record the fluid volume actually used, manufacturer, lot #, expiration date, and start time on Attachment A.

3.4.8 Label the carboy per procedure, and record the labeling data.

3.4.9 Place the carboy containing the dermis tissue and 1M Sodium Chloride into two poly bags, tie a knot in each bag individually to seal close the open end. Place the now bagged carboy in a 190 to 38° C. environment.

3.4.10 The tissue must remain in the Sodium Chloride solution for 18-24 hours.

3.4.11 Record the clean room temperature using a calibrated thermometer. The temperature is to be between 19-38° C.

4.0 Removal of Epidermis by Manual Debridement:

4.0.1 Aseptically transfer the carboy containing the donor material into a class 1000 or better environment.

4.0.2 Record the clean room temperature using a calibrated thermometer. Record the temperature on Attachment A. The temperature is to be between 19-38° C.

4.0.3 Remove the tissue from the carboy and place the dermis onto a sterile cloth, dermis side down.

4.0.4 Using forceps and a blunt dissecting tool, remove the epidermis layer from the exterior surface. Take care not to cut the dermis. Upon removal of the epidermis layer, the dermis should be turned over (epidermis side down) and debrided of extraneous fat or other adherent tissue.

5.0 Microbial Reduction With Benzalkonium Chloride:

5.0.1 While the dermis is still on the sterile cloth, empty the container and thoroughly rinse the container with purified water.

5.0.2 Place debrided dermis tissue back into the carboy.

5.0.3 Fill carboy with at least 10 times the volume of the tissue of filtered 1% benzalkonium chloride. (See attachment A for the minimum predetermined volume). The actual measured amount of fluid is to be recorded on attachment A.

5.0.4 Cap carboy tightly, and invert 3-4 times.

5.0.5 Record the fluid volume, lot # of the 1% benzalkonium chloride and associated expiration date, chemical manufacturer, and start time on Attachment A.

5.0.6 Place the carboy containing the dermis tissue and 1% benzalkonium chloride into two poly bags, tie a knot in the bags and place in a 4°±2° C. environment.

5.0.7 The dermis tissue is to be left in the 1% benzalkonium chloride solution at 4°±2° C. for a period of 1-24 hours.

6.0 Cell Lysis With Hydrogen Peroxide and Tween:

6.0.1 Fill the sonic with purified water to a level 2/3 the height of the selected sonic container (see table 1).

6.0.2 Remove the dermis from the carboy and place into the selected sonic container.

6.0.3 Record the “Time Off” in which the dermis tissue was removed from the 1% benzalkonium chloride solution on Attachment A.

6.0.4 Fill sonic cup with at least 10 times the volume of the tissue with 1% tween −20+0.5% hydrogen peroxide solution. (see attachment A for the minimum predetermined volume)

6.0.5 Record the fluid volume, lot # of the 1% tween −20+0.5% hydrogen peroxide solution and associated expiration date, chemical manufacturer, and start time on Attachment A.

6.0.6 Place the sonic container in sonic for 14±1 minutes.

6.0.7 Turn on the sonic power control.

6.0.8 Stir the dermis at least once every minute. Some foaming may occur. If using a sonic container insert, raise the insert above the level of the fluid and drop down to the resting level. This stirring action should reduce foam levels.

6.0.9 Record the end time on Attachment A.

6.0.10 Remove the sonic container from the sonic device.

6.0.11 Turn off the sonic until the tissue is ready to go back into the sonic.

6.0.12 Record the temperature of the water bath on Attachment A. If the temperature is ≧26° C., then drain and replace the sonic water.

6.0.13 Change gloves prior to touching any tissue after working with the sonic and thermometer.

6.0.14 Pour the 1% tween −20+0.5% hydrogen peroxide solution with the dermis into the sieve over a sink or dump bucket.

6.0.15 Place the dermis on a sterile absorbent material.

7.0 Microbial Reduction With Calcium Hydroxide:

7.0.1 Thoroughly rinse out the sonic container with purified water.

7.0.2 Repeat the rinse step with a small amount of saturated calcium hydroxide solution making sure to coat the entire container.

7.0.3 Place the dermis into a sonication container and fill with at least 10 times the minimum volume of the tissue with saturated calcium hydroxide solution. (See attachment A for minimum predetermined volume.)

7.0.4 Record the fluid volume, lot # of the saturated calcium hydroxide and associated expiration date, chemical manufacturer, and start time on Attachment A.

7.0.5 Turn on sonic device and place the sonic container in sonic for 14±1 minutes. Stir once a minute. If using a sonic container insert, raise the insert above the level of the fluid and drop down to the resting level as the stirring action.

7.0.6 Record the end time on Attachment A.

7.0.7 Remove the sonic container from the sonic.

7.0.8 Turn off the sonic until the tissue is ready to go back into the sonic.

7.0.9 Record the temperature of the water bath on Attachment A. If the temperature is ≧26° C., then drain and replace the sonic water.

7.0.10 Change gloves prior to touching any equipment that comes in contact with tissue after working with the sonic and thermometer.

7.0.11 Pour the solution with the dermis into a sieve over a sink or dump bucket.

7.0.12 Remove dermis from sieve and place it onto a sterile absorbent material.

8.0 Removal of Basal Epithelium and Hair by Manual Debridement:

8.0.1 Using the sterile Polypropylene scour wipe, vigorously bush both sides of the dermis to remove any hair or epithelium.

8.0.2 Perform a visual inspection of the dermis.

8.0.2.1 Dermis must be free of hair, tears, holes, cuts, and transparent areas.

9.0 Rinse (2×) to Remove Calcium Hydroxide:

9.0.1 Thoroughly Rinse the sonic container with purified water.

9.0.2 Place the dermis in the sonic container. Fill with at least 10 times the minimum volume of the tissue with purified water on the dermis and gently agitate using a swirling motion for 4±1 minutes. (See attachment A for minimum predetermined volume.) If using a sonic container insert, raise the insert above the level of the fluid and drop down to the resting level as the stirring action. Continuously agitate during this step, but do not sonicate.

9.0.3 Pour the solution with the dermis into the sieve over a sink or dump bucket.

9.0.4 Repeat steps 9.0.2-9.0.3 one more time.

9.0.5 Remove the dermis from sieve and place onto a sterile absorbent material.

10.0 Chelation of Calcium With EDTA:

10.0.1 Rinse the sonic container with a small amount of 0.1% EDTA solution making sure to coat the entire container.

10.0.2 Place the dermis in the sonic container and fill with at least 10 times the volume of the tissue with 0.1% EDTA solution. (see attachment A for minimum predetermined volume) This step is to remove the calcium.

10.0.3 Record the fluid volume, Lot # of the 0.1% EDTA solution and associated expiration date, chemical manufacturer, and start time on Attachment A.

10.0.4 Turn on the sonic device.

10.0.5 Place the sonic container in sonic for 14÷1 minutes. Continuously agitate by stirring or by using the sonic container insert during this step. If using a sonic container insert, raise the insert above the level of the fluid and drop down to the resting level as the stirring action.

10.0.6 Record the end time on Attachment A.

10.0.7 Remove the sonic container from the sonic.

10.0.8 Turn off the sonic.

10.0.9 Record the temperature of the water bath on Attachment A. If the temperature is ≧26° C., then drain and replace the sonic water.

10.0.10 Change gloves prior to touching any equipment that comes in contact with tissue after working with the sonic and thermometer.

10.0.11 Pour the solution with the dermis into the sieve over a sink or dump bucket.

10.0.12 Remove dermis from sieve and place onto a sterile absorbent material.

11.0 Rinse (2×) to Remove EDTA:

11.0.1 Rinse the sonic container with purified water.

11.0.2 Place the dermis in the sonic container. Fill with at least 10 times the minimum volume of the tissue with purified water on the dermis and gently agitate using a swirling motion for 4±1 minutes. (see attachment A for minimum predetermined volume) If using a sonic container insert, raise the insert above the level of the fluid and drop down to the resting level as the stirring action. Continuously agitate during this step, but do not sonicate.

11.0.3 Pour the solution with the dermis into the sieve over a sink or dump bucket.

11.0.4 Repeat steps 11.0.2-11.0.3 one more time.

11.0.5 Remove dermis from sieve and place onto a tex-wipe.

12.0 Neutralization of Dermis pH With Buffer:

12.0.1 Rinse the sonic container with a small amount of pH 7.0 buffer solution making sure to coat the entire container.

12.0.2 Place the dermis in the sonic container and fill with at least 10 times the volume of the tissue of the tissue with pH 7.0 buffer solution. (See attachment A for minimum predetermined volume.)

12.0.3 Record the fluid volume, Lot # of the pH 7.0 buffer solution and associated expiration date, chemical manufacturer, and start time on Attachment A.

12.0.4 Gently agitate for 14±1 minutes. If using a sonic container insert, raise the insert above the level of the fluid and drop down to the resting level as the stirring action.

12.0.5 Record the end time on Attachment A.

12.0.6 Pour the solution with the dermis into the sieve over a sink or dump bucket.

12.0.7 Remove dermis from sieve and place onto sterile absorbent material.

13.0 Rinse (3×) to Remove Buffer:

13.0.1 The dermis is to be spread out on the sieve and sprayed front and back with purified water. The tissue is to be sprayed as to have saturated all of the tissue with the purified water.

13.0.2 Rinse the sonic container with purified water.

13.0.3 Place the dermis in the sonic container. Fill with at least 10 times the minimum volume of the tissue with purified water on the dermis and gently agitate using a swirling motion for 4±1 minutes. (See attachment A for minimum predetermined volume.) If using a sonic container insert, raise the insert above the level of the fluid and drop down to the resting level as the stirring action. Continuously agitate during this step, but do NOT sonicate.

13.0.4 Pour the solution with the dermis into the sieve over a sink or dump bucket.

13.0.5 Repeat steps 13.02-13.03 two more times.

13.0.6 ½ fill a sterile centrifuge tube with the last rinse water and test the pH to ensure that it is over 5.5 and below 8.0.

13.0.7 Remove dermis from sieve and place onto a sterile absorbent material.

14.0 Drying the Dermis With Isopropanol:

14.0.1 Repeat rinse with a small amount of 70% isopropanol making sure to coat the entire container.

14.0.2 Place the dermis in the sonic container and fill with at least 10 times the volume of the tissue of 70% isopropanol. (See attachment A for minimum predetermined volume.)

14.0.3 Record the fluid volume, lot # of the 70% isopropanol and associated expiration date, chemical manufacturer, and start time on Attachment A.

14.0.4 Gently agitate for 14±1 minutes. If using a sonic container insert, raise the insert above the level of the fluid and drop down to the resting level as the stirring action. Continuously agitate during this step.

14.0.5 Record the end time on Attachment A.

14.0.6 Pour the solution with the dermis into the sieve over a sink or dump bucket.

14.0.7 Place each piece of dermis between two pieces of folded sterile absorbent material (e.g., “tex-wipes”) and press to dry the tissue as completely as possible. Ensure that the dermis is as flat as possible.

14.0.8 The dermis is to be laid flat in the lyophilization bag and sealed as not to allow the tissue to fall out of the bag. If required, the tissue may be cut prior to placement into a lyophilization bag Try to maintain the tissue in a flat single layer in the lyophilization bag.

14.0.9 Label each lyophilization bag with the donor number, the date, and the processor initials.

14.0.10 Place the Lyophilization bags containing the dermis tissue into two poly bags; tie a knot in each bag individually to seal close the open end.

14.0.11 Place into the appropriate freezer for staging to go into lyophilization. When placing the bagged dermis into a freezer, lay it as flat as possible.

14.0.11.1 Record the freezer location on Attachment A.

15.0 Lyophilization:

15.0.1 Lyophilize. Operate the freeze dryer under standard conditions. Using a screen press, ensure that the tissue is pressed flat upon placement into the freeze dryer, prior to starting the unit.

15.0.2 Write the lyophilization run number on the bag prior to exiting the lyophilization area.

15.0.3 Attach the lyophilization data to the production record and initial and date on Attachment A.

15.0.4 After completion of the freeze drying, place the tissue in the dermis freeze-dried staging cabinet. Write on Attachment A the location of the tissue post lyophilization.

16.0 Cutting, Packaging, and Sampling:

16.0.1 “Pass In” the intermediate product bar code from the freeze dryer (lyophilizer).

16.0.2 Perform a visual inspection of the dermis. Dermis must be white, off-white, or tan with slight cast of pink or gray and no discolored patches.

16.0.3 An independent verification of Quality Control visual inspection criteria must be performed. Personnel conducting independent verification shall record information appropriately on attachment A.

16.0.4 Cut grafts according to specification for dermis grafts.

16.0.5 Perform a final visual inspection of the finished grafts: The grafts must be clean cut. (No ragged edges or excess tissue pieces remaining attached to the edges.)

16.0.6 Re-measure the grafts to ensure the grafts are to specifications.

16.0.7 Record these measurements, swab the grafts, and package material.

16.0.8 Label each graft.

17.0 Irradiation:

17.0.1 Pack according to standard arrangement for irradiation.

17.0.2 Ship to Irradiation Facility.

17.0.3 Receive back from Irradiation.

17.0.4 Attach a copy of irradiation certificate to Allograft production record.

It is noted that the process can be stopped after any one of the following steps: Removal of Epidermis with Sodium Chloride, Microbial Reduction with Benzalkonium Chloride, Cell Lysis with Hydrogen Peroxide and Tween, if the Dermis is rinsed appropriately. The process can also be stopped after the step, Rinse (3×) to Remove Buffer. The dermis can be doubled bagged, labeled with the donor #, the last step processed and frozen.

Attachment A

| | | | |
|---|---|---|---|
| | | Room | Completed |
| Step | Description | Number | By and Dated |

| | |
|---|---|
| 1 | Evaluation of Tissue and 1M Sodium Chloride (18-24 hours. @ 19-38° C.) |
| | Thickness Gauge # _{———————} |
| | Contact check measurement point 1: _{————}, 2: _{————}, |

3: _{————}, 4: ————

| | |
|---|---|
| | Measure the tissue. Use Attachment B |
| | Lot# _{—————} Mfg. _{—————} Exp. Date _{—————} |
| | Volume of tissue _{———} × 10 ml = _{———} volume of fluids to be used. |
| | Time on 1 M Sodium Chloride: _{———————} |
| | Bar code passed out to incubator # _{———}: |
| | Volume of 1 M Sodium Chloride used: _{———————} |
| | Temperature of room: _{————} ° C. |
| * | Room cleaned and disinfected |
| 2a | Time of 1 M Sodium Chloride: _{————————} |
| 2b | Debride |
| | Bar code passed in from incubator: |
| | Temperature of room: _{————} ° C. _{—————} |
| | Visual Inspection by independent individual initials |
| 3 | 1% Benzalkonium Chloride (1-24 hours. @ 4 ± 2° C.) |
| | Lot# _{—————} Mfg. _{—————} Exp. Date _{—————} |
| | Bar code passed out to refrigerator: |
| | Time on 1% Benzalkonium Chloride: _{————————} |
| | Volume of 1% Benzalkonium Chloride used: _{————————} |
| * | Room cleaned and disinfected |
| 4a | Temperature of room: _{————} ° C. |
| | Time off 1% Benzalkonium Chloride: _{————————} |
| 4b | 1% Tween-20 +0.5% H₂O₂ (+sonication 14 ± 1 min. @ 19-30° C.) |
| | Lot# _{—————} Mfg. _{—————} Exp. Date _{—————} |
| | Volume of 1% Tween-20 +0.5% H₂O₂ used: _{————————} |
| | Bar code passed in from refrigerator: |
| | Start Time: _{—————} End time: _{—————} End temperature: _{—————} ° C. |
| 5 | Saturated Ca Hydroxide (+sonication 14 ± 1 mm. @ 19-30° C.) |
| | Lot# _{—————} Mfg. _{—————} Exp. Date _{—————} |
| | Volume of Saturated Ca Hydroxide used: _{————————} |
| | Start time: _{—————} End time: _{—————} End temperature: _{—————} ° C. |
| 6 | Debride |
| 7a | Water rinse (4 ± 1 min. @ 19-30° C.) Volume of water used: _{———} |
| | Lot# _{—————} Mfg. _{—————} Exp. Date _{—————} |
| 7b | Water rinse (4 ± 1 min. @ 19-30° C.) Volume of water used: _{————} |
| 8 | 1% EDTA (+sonication 14 ± 1 min. {fourth root} 19-30° C.) |
| | Lot# _{—————} Mfg. _{—————} Exp. Date _{—————} |
| | Volume of Saturated 1% EDTA used: _{————————} |
| | Start time: _{—————} End time: _{—————} End temperature: _{—————} ° C. |
| 9a | Water rinse (4 ± 1 min. @ 19-30° C.) Volume of water used: _{————} |
| 9b | Water rinse (4 ± 1 min. @ 19-30° C.) Volume of water used: _{————} |
| 10 | Buffer Wash (14 ± 1 min. @ 19-30° C.) |
| | Lot# _{—————} Mfg. _{—————} Exp. Date _{—————} |
| | Volume of buffer wash used: _{————————} |
| | Start time: _{—————} End time: _{—————} |
| 11a | Water rinse (4 ± 1 min. @ 19-30° C.) Volume of water used: _{————} |
| 11b | Water rinse (4 ± 1 min. @ 19-30° C.) Volume of water used: _{————} |
| 11c | Water rinse (4 ± 1 min. @ 19-30° C.) |
| | Volume of water used: _{————— pH of last water rinse} _{————} |
| 12 | 70% Isopropyl Alcohol (14 ± 1 min. @ 19-30° C.) |
| | Lot# _{—————} Mfg. _{—————} Exp. Date _{—————} |
| | Volume of 70% Isopropyl Alcohol used: _{————————} |
| | Start time: _{—————} End time: _{—————} |
| | Number of bags in freezer #_{—————} |
| | Bar code passed out to freezer #_{—————} (pre-lyo |
| * | Room cleaned and disinfected |
| 13 | Lyo data attached, Post Lyo location _{———————} |
| | Number of bags going into post Lyo location # _{—————} |
| 14a | Cut and Seal |
| | Temperature of room: _{————} ° C. Thickness Gauge #: _{————} |
| | Final Contact check measurement point 1: _{————}, 2: _{————}, |

3: _{————}, 4: _{————}

Template #'s: _{——————}, _{——————},

: _{——————}, _{——————},
: _{——————}, _{——————}.

| | |
|---|---|
| | Independent visual inspection initials: _{——————————} |
| | Bar code passed in (post lyo): |
| 14b | Graft number and size: MF0001 attached |
| 15 | |
| 16a | |
| | Sealer # _{———Alarm Active □} |
| | OR 100% Inspection by Auditor _{————} |
| | # of total grafts packaged _{————} # of Samples to test: _{————} |
| | # of sample Passes: _{————} # of Samples Failures: _{————} |
| | Testing Person: _{——————————} |
| * | Room cleaned and disinfected |
| 17 | Irradiation Certificate attached |
| 18 | Enzyme Lability Sample Submitted |

Manufacturing Review by: _{——————————} Date: _{————}
Data Entered by: _{——————————} Date: _{————}
QCReview: _{——————————} Date: _{————}
The disclosure of all patents and publications cited in this application are incorporated by
reference in their entirety to the extent that their teachings are not inconsistent with the teachings herein. It
should be understood that the examples and embodiments described herein are for illustrative purposes
only and that various modifications or changes in light thereof will be suggested to persons skilled in the art
and are to be included within the spirit and purview of this application and the scope of the appended
claims.

What is claimed is:

1. A method of processing collagen-based tissue prior to implantation into a recipient in need thereof, said method comprising the steps of:
decellurizing said collagen-based tissue such that substantially all cells, cellular debris, lipids and proteins are removed; and
preserving the resulting collagen scaffold through a bioreactor, cryopreservation, freezing, chilling, drying, room temperature packaging, or freeze-drying.

2. The method of processing collagen-based tissue prior to implantation into a recipient in need thereof, according to claim 1, further comprising repopulating the collagen scaffold with cells having lower immunogenicity toward the recipient than the collagen-based tissue; and growing said cells on and within said collagen-based tissue in an organ perfusion system.

3. An acellular collagen-based tissue produced according to the method of claim 1.

4. The method according to claim 1, wherein said collagen-based tissue is selected from the group consisting of a heart, heart valve, joint, soft tissue organ and vasculature.

5. The method according to claim 1, wherein said collagen-based tissue consists of a total joint.

6. The method according to claim 1, wherein said collagen-based tissue consists of a trachea.

7. The method according to claim 1, wherein said collagen-based tissue consists of a knee, shoulder, wrist, ankle or elbow joint.

8. A method of replacing collagen-based tissue with a processed collagen-based tissue in a recipient in need thereof which comprises implanting acellular collagen-based tissue or acellular collagen-based tissue repopulated with cells into said recipient.

9. An implant cleaning, perfusion and passivation process which comprises cyclic exposure of said implant to increased and decreased positive or negative pressures, or both.

10. An apparatus for conducting the process according to claim 9 comprising a reaction chamber 120, said reaction chamber 120 in communication with an air piston 110, such that increased pressure and decreased positive or negative pressures, or both, via movement of the air piston 110.

11. The apparatus of claim 10 wherein said air piston 110 and said reaction chamber 120 are connected via a conduit 128.

12. The apparatus of claim 12, wherein said apparatus comprises a filter 122 along the conduit 128.

13. A method for treating and processing tissue for implantation that decellularizes and inactivates virus in said tissue comprising the steps of:
a) contacting said tissue with a viral inactivating agent, wherein said viral inactivating agent comprises benzalkonium chloride; and
b) contacting said tissue with a decellularizing agent; whereby said tissue maintains structural integrity and activity of growth factors in said tissue is maintained.

14. The method of claim 13, wherein said viral inactivating agent comprises about 0.5 percent or more, weight percent, benzalkonium chloride solution.

15. The method of claim 14, wherein said viral inactivating agent comprises about 0.5 percent, weight percent, benzalkonium chloride solution.

16. The method of claim 13, wherein said decellularizing agent comprises a solution comprising, by weight, about 0.5 percent or more Tween 20 and about 0.5 percent or more hydrogen peroxide.

17. The method of claim 16, wherein said decellularizing agent comprises about 1 percent Tween 20 and about 0.5 percent hydrogen peroxide, and wherein said tissue is sonicated during contact with said decellularizing agent.

18. The method of claim 13, wherein said tissue is bone, neural tissue, fibrous connective tissue including tendons and ligaments, cartilage, dura, pericardia, muscle, heart valves, veins and arteries and other vasculature, dermis, adipose tissue, or glandular tissue.

19. The method of claim 18 wherein said tissue is bone, heart valve(s), vein(s), tendon, ligament or dermis.

20. The method of claim 13 wherein said tissue is dermis.

21. A method of decellularizing and viral inactivating tissue comprising the steps of:
a) contacting said tissue with a viral inactivating agent; and
b) contacting said tissue with a decellularizing agent, wherein said decellularizing agent comprises a solution comprising, by weight, about 0.5 percent or more Tween 20 and about 0.5 percent or more hydrogen peroxide.

22. The method of claim 21 wherein said decellularizing agent comprises a solution comprising, by weight, about 1 percent tween 20 and about 0.5 percent hydrogen peroxide; and wherein said method further optionally comprises sonicating said tissue during step b.

23. The method of claim 21 wherein said tissue is dermis.

24. A method for treating tissue effecting the decellularizing and inactivating viruses in said tissue comprising the steps of:
a) contacting said tissue with a solution comprising about 0.5 percent or more, by weight, benzalkonium chloride;
b) contacting said tissue with a solution comprising about 0.5 percent or more, by weight, tween 20 and about 0.5 percent or more, by weight, hydrogen peroxide; and
c) contacting said tissue with a calcium hydroxide solution.

25. The method of claim 24, wherein said calcium hydroxide solution is saturated.

26. The method of claim 24, further comprising contacting said tissue treated with said calcium hydroxide solution with a calcium chelating agent; optionally sonicating said tissue during contacting said tissue with said chelating agent.

27. The method of claim 26, wherein said calcium chelating agent is a solution comprising about 0.5 percent to about 5 percent EDTA.

28. The method of claim 24, further comprising drying said tissue.

29. The method of claim 28 wherein drying said tissue comprises contacting said tissue with an alcohol solution.

30. The method of claim 24, further comprising lyophilizing said tissue.

31. The method of claim 24, further comprising cutting and packaging said tissue.

32. The method of claim 24, wherein said tissue is sonicated during steps b and c.

33. The method of claim 32 further comprising irradiating said tissue.

34. A method for decellularizing and inactivating viruses in dermis tissue comprising the steps of:
a) obtaining a sample of crude dermis tissue;
b) treating said crude dermis tissue with sodium chloride;
c) separating epidermis from dermis of said crude dermis tissue by manual debridement to produce dermis sample;
d) contacting said dermis sample with a solution comprising 0.5 percent or more, by weight, benzalkonium chloride;
e) contacting said dermis sample with a solution comprising 0.5 percent or more, by weight, tween 20 and 0.5 percent or more hydrogen peroxide; optionally further comprising simultaneous sonication of said dermis sample;
f) contacting said dermis with a solution of saturated calcium hydroxide; and subsequent rinsing of said dermis sample followed by chelating of said dermis sample by contact with a chelating agent; and optionally further comprising sonicating said dermis sample during contact with said saturated calcium hydroxide;
g) neutralizing pH of said dermis sample with a neutralizing buffer, followed by rinsing said dermis sample
h) drying said dermis sample with an alcohol solution comprising about 50 to about 100 percent, by weight, alcohol;
i) lyophilizing said dermis sample;
j) cutting said dermis sample; and
k) irradiating said dermis sample.
